(19) United States
(12) Patent Application Publication
(10) Pub. No.: US 2012/0328581 A1
(43) Pub. Date: Dec. 27, 2012

(54) TISSUE REPAIR

(75) Inventor: Mike LEEK, Manchester (GB)

(73) Assignee: Intercytex Limited, Wythenshawe (GB)

(21) Appl. No.: 13/548,964

(22) Filed: Jul. 13, 2012

Related U.S. Application Data
(63) Continuation of application No. 12/301,851, filed on Apr. 8, 2009.

(30) Foreign Application Priority Data
May 25, 2006 (GB) 0610414.5

(51) Int. Cl. A61K 35/36; A61P 17/02; A61P 17/00

(52) U.S. Cl. 424/93.7

(57) ABSTRACT

According to the invention there is provided a cosmetic method for the augmentation of subcutaneous or dermal tissue in a subject, which method comprises the steps of:

(i) providing a suspension of autologous dermal fibroblasts; and

(ii) injecting an effective volume of the suspension into tissue subadjacent to the subcutaneous or dermal tissue so that the tissue is augmented;

wherein the fibroblasts are suspended in HYPOTHERMOSOL®, preferably HYPOTHERMOSOL®FRS.

### CROSS-REFERENCE TO RELATED APPLICATIONS

This application is a continuation of U.S. application Ser. No. 12/301,851, filed Nov. 21, 2008, which is the U.S. National Stage filing under 35 U.S.C. §371 of International Application No. PCT/GB/2007/001910, filed May 23, 2007, which claims benefit of United Kingdom Application No. 0610414.5, filed May 25, 2006.

The present invention relates to the repair of skin and soft tissue defects, including wrinkles, particularly by injecting materials that augment the volume of the dermis or subcutaneous tissue in human subjects.

Injectable materials have been extensively used for correcting skin and soft tissue defects, especially facial soft tissue defects (see review by Homicz & Watson, 2004, Facial Plastic Surgery 20: 21-29). Defects which respond well to injectable augmentation include static facial rhytids of the forehead, glabella, perioral region, and lateral periorbital (crow's feet) area. An advantage of injectable materials over traditional surgical techniques include ease of administration and minimal pain and discomfort for a subject being treated. Treatment can be conducted in an office setting, thereby reducing costs. However, injectable materials are often reabsorbed over time, which means that repeated applications may be need to achieve the desired results. Within the first 72 h after an injection, the treated area may exhibit transient erythema, edema, ecchymosis and induration. Hypersensitivity reactions may also occur, the severity of which depends on the material being injected and the subject's immune system.

Materials used for injection as dermal augmentation include synthetic and biological materials, with the latter being categorised as xenografts (where the donor and the subject are different species), autografts (where the donor and the subject are the same individual), or allografts (where the donor and the subject are different individuals but the same species; also known as homografts).

The development of synthetic materials for repair of skin and soft tissue defects can be traced back to the use of paraffin in the early 1900s for elevation of facial furrows and depressed contours. The development of purified fluid silicone in the 1950s encouraged further interest in tissue augmentation by synthetic materials. Injection of medical-grade fluid silicone provided a means for firming tissue in such a way as to mimic the supple texture of normal tissue. However, there were several problems with injectable silicone, such as unpredictable inflammatory reaction, migration, extrusion, ulceration, silicone granuloma formation, and even organ failure and death in cases of granulomatous hepatitis, pulmonary embolism and silicone pneumonitis. Although still used in various countries around the world, the FDA declared the use of injectable silicone illegal in 1991.

Synthetic materials are considered to be desirable for tissue augmentation due their potential longevity, and other more reliable and promising synthetic materials are available or are being developed. However, longevity may be a problem if a subject is dissatisfied with the cosmetic effects, if there are adverse tissue reactions to the implanted materials, or if a subject wishes to change their appearance due to changes in fashion trends. As of 2004, no injectable synthetic materials were approved by the US Food and Drug Administration (FDA) for dermal augmentation (Homicz & Watson, 2004, supra).

The most widely used dermal filler is the xenograft biological material bovine collagen. Commercial products comprise purified, enzyme-digested collagen (predominantly type I, with less than 5% type III collagen), and are available as ZYDERM® and ZYPLAST® from Inamed Aesthetics (Santa Barbara, Calif., USA). ZYDERM® is processed to remove by enzymatic degradation the C- and N-terminal peptides of bovine collagen, which are immunogenic in human subjects, thereby yielding a material called atelocollagen that can be used in pre-screened non-reactive subjects (see U.S. Pat. No. 3,949,073; U.S. Pat. No. 4,424,208; and U.S. Pat. No. 4,488,911). The ZYDERM® products are reported to be prone to loss of volume over time due to absorption by the host. To address this problem, a glutaraldehyde cross-linked atelocollagen called ZYPLAST® was developed (see U.S. Pat. No. 4,582,640; and U.S. Pat. No. 4,642,117). However, some investigators report that there is no or little increased persistence of ZYPLAST® compared to ZYDERM® (Matti & Nicolle, 1990, Aesthetic Plastic Surgery 14: 227-234; Ozgentas et al., 1994, Ann Plastic Surgery 33: 171).

Following two decades of clinical experience and with the advantages of demonstrated safety and reliability, bovine collagen is regarded as the gold standard for tissue augmentation.

Disadvantages of bovine collagen include limited duration of effect, and thus the need for multiple treatments, and the potential for allergic reaction.

Hyaluronic acid is a glycosaminoglycan macromolecule found in the native extracellular matrix of connective tissue. The macromolecule is composed of chains of repeating disaccharide units and due to its hydrophobic structure attracts water into the extracellular matrix to provide turgor to connective tissue. Although hyaluronic acid is identical in structure across different species, thereby reducing the possibility of antigenic cross-reactivity in a host, it undergoes local degradation after transplantation. Commercially available products, including the HYLAFORM® range developed by Genzyme Biosurgery and RESTYLANE® range developed by Q-Med Corporation (Uppsala, Sweden), are cross-linked gels of hyaluronic acid derivatives from rooster comb (HYLAFORM®) or from a streptococcal fermentation process (RESTYLANE®). Although the incidence of hypersensitivity to these hyaluronic acid derivatives appears to be low, subjects are still at some risk of allergic reaction. Furthermore, as with bovine collagen, hyaluronic acid derivatives provide a limited duration of effect.

Materials which have been used in autografts for the repair of skin and soft tissue defects include autologous fat, autologous dermal extracellular matrix extract (AUTOLOGEN®) and autologous dermal fibroblasts (ISOLAGEN®). Liposuction techniques developed in the 1970s provided an effective means for harvesting adipose tissue (fat) from a subject which could then be injected back into the subject in the target region. Although autologous fat grafting has the advantage of minimal risk of allergic reaction and bioincompatibility, it requires a donor site on the subject and has an unpredictable resorption rate which means that it is difficult to easily correct skin and soft tissue defects with precision.

AUTOLOGEN® (developed by Collagenesis Corporation, Beverly, Mass., USA) comprises a dermal extracellular matrix isolated from a subject's skin. The subject's skin, once excised, is processed to isolate dermal matrix components including collagen (types I, III and VI), elastin, fibronectin and glycosaminoglycans. A suspension of these components is then used for injection into a subject for soft tissue augmentation. Treatment with AUTOLOGEN® requires a significant volume of a subject's skin to produce the injectable suspension, and repeated injections are often required to maintain the aesthetic effect of the treatment.

U.S. Pat. No. 5,591,444 describes a method for repairing subcutaneous or dermal tissue in a subject by injection of a suspension of autologous dermal fibroblasts (“ISOLAGEN®”, developed by Isolagen Technologies, Houston, Tex., USA) into the dermis and subcutaneous tissue subadjacent to the defect. The method involves the preparation of autologous cultured dermal fibroblasts from a specimen obtained from the subject and subsequent injection of the fibroblast preparation into the subject. Initial results regarding the longevity of effect of the treatment have been encouraging, and further advantages include minimisation of risk of allergic reaction and bioincompatibility due to the autologous nature of the injected material. A disadvantage of the use of autologous cells is that they have not been shown to have a particularly long viability period (“shelf life”) after they have been cultured. For example, it is believed that the autologous cells according to U.S. Pat. No. 5,591,444 need to be implanted in the donor patient within about 8 hours after the end of the culture period. This means that it is necessary to arrange the logistics of treatment so that the cultured autologous cells and the donor/patient are available in the same location at the same time.

In summary, none of the above-mentioned available injectable materials is wholly satisfactory for the purpose of augmenting the subadjacent dermis and soft tissue, and the search for a successful, reliable and cost-effective material continues.

According to a first aspect of the invention there is provided a cosmetic method for the augmentation of subcutaneous or dermal tissue in a subject, which method comprises the steps of:
(i) providing a suspension of autologous dermal fibroblasts; and
(ii) injecting an effective volume of the suspension into tissue subadjacent to the subcutaneous or dermal tissue so that the tissue is augmented, wherein the fibroblasts are suspended in HYPOTHERMOSOL®, preferably HYPOTHERMOSOL® FRS.

The method preferably has a long-term effect (for example, longer than 4 to 6 months or a year).

In an embodiment of the first aspect of the invention, the method further comprises the steps of a) obtaining an autologous dermal biopsy from the subject; b) passaging the dermal fibroblasts from the dermal biopsy in a culture medium comprising between 0.5% and 20% non-human serum, so as to provide dermal fibroblasts substantially free of adipocytes, keratinocytes and extracellular matrix; and c) forming a suspension of the passaged fibroblasts. For example, the suspension is formed by scraping the fibroblasts and/or exposing the fibroblasts to a proteolytic enzyme and/or an animal product free alternative such as NO-ZYME™. In another embodiment, the method further comprises the in vitro steps of a) obtaining dermal fibroblasts from an autologous dermal biopsy sample obtained from the subject; b) passaging the dermal fibroblasts from the dermal biopsy in a culture medium comprising between 0.5% and 20% non-human serum, so as to provide dermal fibroblasts substantially free of adipocytes, keratinocytes and extracellular matrix; and c) forming a suspension of the passaged fibroblasts. For example, the suspension is formed by scraping the fibroblasts and/or exposing the fibroblasts to a proteolytic enzyme and/or an animal product free alternative such as NO-ZYME™. Further aspects of the invention are recited in the accompanying claims.

The invention also relates to a composition which comprises autologous dermal fibroblasts suspended in HYPOTHERMOSOL®, preferably HYPOTHERMOSOL® FRS.

Typical defects that can be corrected by this present invention include rhytids, stretch marks, depressed scars, cutaneous depressions of non-traumatic origin, scaring from acne vulgaris, and hypoplasia of the lip. The cells that are injected or applied, according to the invention, are autologous cells preferably expanded by passage in a cell culture system.

The suspension of dermal fibroblasts is in one embodiment substantially free of immunogenic proteins.

The present invention is based on an improvement of the invention involving the use of autologous fibroblasts from a subject as described in U.S. Pat. No. 5,591,444 for the repair of skin and soft tissue. Where legally permissible the content of U.S. Pat. No. 5,591,444 is incorporated herein by reference in its entirety.

Cells for application (for example, by injection) are suspended in a delivery medium which is HYPOTHERMOSOL®, preferably HYPOTHERMOSOL® FRS.

HYPOTHERMOSOL®, preferably HYPOTHERMOSOL® FRS (Registered Trade Mark BioLife Solutions, Inc.) is a preservation medium manufactured by BioLife Solutions, Inc. of Owega, N.Y. 13827 (www.BioLifeSolutions.com)

The components of HYPOTHERMOSOL® FRS are shown in Table 1:

TABLE 1
HYPOTHERMOSOL ® FRS Composition List

| Components | |
|---|---|
| Trolox | |
| Na⁺ | |
| K⁺ | |
| Ca²⁺ | |
| Mg²⁺ | |
| Cl⁻ | |
| H₂PO₄⁻ | |
| HCO₃⁻ | |
| HEPES | |
| Lactobionate | |
| Sucrose | |
| Mannitol | |
| Glucose | |
| Dextran-40 | |
| Adenosine | |
| Glutathione | |
| pH | 7.6 |
| Osmolality | ~360 |

The volume of saline or delivery medium in which the cells are suspended depends upon such factors as the number of fibroblasts the practitioner desires to inject, the size and number of the defects that are to be treated and the urgency of the subject's desire to obtain the results of treatment. The practitioner can thus suspend cells in a larger volume of saline or medium and inject correspondingly fewer cells at each injection site if required.

For example, according to the invention, between 10⁴ and 10⁸, or between 10⁶ and 10⁸, preferably about 1-5×10⁷, for example 1×10⁷ or 4×10⁷, fibroblasts are injected. For example, according to the invention, a volume of about 1 ml of suspension is injected. A volume of 0.8 ml maybe used.

It has been found that the commercially available product HYPOTHERMOSOL®, particularly HYPOTHERMOSOL® FRS, is useful in preserving the cultured cells in a viable condition prior to administration to a patient.

The cultured fibroblast cells are at a state of high metabolic activity and have a high energy demand. Under normal conditions they may tend to suffer apoptosis, particularly when they are suspended in a medium at relatively high cell density. It appears that suspension of the cells in HYPOTHERMOSOL® allows the cells to remain viable and alive for a longer period than would be the case without this medium. Further, whilst cooling of any viable population of cells in a medium would be expected to prolong viability/life; the use of HYPOTHERMOSOL® with cultured fibroblast cells, together with cooling, appears to provide an unexpectedly advantageous preservation effect; this results in an important benefit of elongated “shelf life” for the product.

As a further aspect of the invention it may be advantageous to suspend the cells for injection in cooled HYPOTHERMOSOL® for a period of time prior to injection. For example, cells may be suspended for conditioning in cooled HYPOTHERMOSOL® for at least an hour at about 2° C. to about 8° C. Subsequently the conditioned cells may be maintained cooled until use, or may be allowed to warm before storage before use. A period of cooled conditioning in HYPOTHERMOSOL® appears to provide a benefit of preservation which can continue even if the material is subsequently warmed. The use of uncooled (ie room temperature) HYPOTHERMOSOL® for conditioning is not desirable. By way of example, cells treated according to this aspect of the invention may be stored, and remain viable, for a period of about 10 days at about 2° C. to about 8° C.

Cell suspensions of the invention can be used to treat dermal defects using the same techniques that those skilled in art presently employ to use ZYDERM® and ZYPLAST®. The cell suspension can be used in place of atelocollagen solutions with the advantages set forth as above. Representative teachings concerning the use of injectable material for augmenting the subadjacent dermis and subcutaneous tissue can be found in the surgical literature (see Gonzales, 1992, Aesthetic Plastic Surgery 16: 231-234; Nicolle, 1985, Aesthetic Plastic Surgery 9: 159-162; & Pieyre, 1985, Aesthetic Plastic Surgery 9: 153-154; which are hereby incorporated by reference in their entirety). The treatment of fine superficial facial lines, one embodiment of the invention, can be accomplished as follows. The area to be treated is prepped with alcohol and stretched to give a taut surface. A syringe is filled with a cell suspension and fitted with a 30 ga. needle for injection. The needle is inserted into the skin site as superficially as possible; the orientation of the bevel is not critical. An intradermal injection is made by gentle pressure until a slight blanch is seen. Multiple serial injections are made.

In other embodiments the injectate can be placed in the obicularis musculature, to treat hypoplasia of the lip or into the subcutaneous tissue to treat deep subcutaneous defects.

Depending on the target area to be treated and/or the viscosity of the cell suspension, a larger or narrower gauge needle than 30 ga. may be used.

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing description. Such modifications are intended to fall within the scope of the appended claims. All cited references are, hereby, incorporated by reference.

1. A method for the repair of a skin defect and/or soft tissue defect in a subject, which method comprises the steps of:
(i) providing a suspension of autologous dermal fibroblasts; and
(ii) injecting an effective volume of the suspension into tissue subadjacent to the skin defect and/or soft tissue defect so that the tissue is augmented, thereby repairing the skin defect and/or soft tissue defect,
wherein the fibroblasts are suspended in a preservation medium having a pH of 7.6 and osmolality of ˜360, and comprising the following components: 6-hydroxy-2,5,7,8-tetramethylchromane-2-carboxylic acid, Na⁺, K⁺, Ca²⁺, Mg²⁺, Cl⁻, H₂PO⁴⁻, HCO³⁻, HEPES, Lactobionate, Sucrose, Mannitol, Glucose, Dextran-40, Adenosine and Glutathione.

2. The method of claim 1 further comprising a step of identifying a defect that is susceptible to amelioration by augmentation of the subadjacent subcutaneous or dermal tissue.

3. The method of claim 1, in which the fibroblasts are passaged.

4. The method of claim 1, wherein the defect is a rhytid, stretch mark, a depressed scar, a cutaneous depression of non-traumatic origin or an under-development of the lip.

5. The method of claim 1, wherein the suspension further comprises fibrin in an amount effective to form an injectable.

6. The method of claim 5, wherein the fibrin is at a final concentration of 0.1 to 20 mg/ml.

7. The method of claim 5, wherein the fibrin is human.

8. The method of claim 1, wherein the suspension further comprises collagen and/or hyaluronic acid.

9. The method of claim 1, which further comprises the steps of:
a) obtaining an autologous dermal biopsy from the subject;
b) passaging the dermal fibroblasts from the dermal biopsy in a culture medium comprising between 0.5% and 20% non-human serum, so as to provide dermal fibroblasts substantially free of adipocytes, keratinocytes and extracellular matrix; and
c) forming a suspension of the passaged fibroblasts.

10. The method of claim 1, which further comprises the in vitro steps of:
a) obtaining dermal fibroblasts from an autologous dermal biopsy sample obtained from the subject;
b) passaging the dermal fibroblasts from the dermal biopsy in a culture medium comprising between 0.5% and 20% non-human serum, so as to provide dermal fibroblasts substantially free of adipocytes, keratinocytes and extracellular matrix; and
c) forming a suspension of the passaged fibroblasts.

11. The method of claim 9, in which the suspension is formed by scraping the fibroblasts and/or exposing the fibroblasts to a proteolytic enzyme and/or an animal product free alternative

12. The method of claim 1, in which the subject is human.

13. The method of claim 1 wherein between 10⁴ and 10⁸ fibroblasts are injected.

14. The method of claim 1 wherein a volume of about 1 ml of suspension is injected.

15. The method of claim 1, wherein the fibroblasts are suspended in the preservation medium at a temperature of about 2° C. to about 8° C. for at least one hour.

16. The method of claim 1 wherein
a) the fibroblasts are suspended in the preservation medium; and
b) about 1×10⁷ or 4×10⁷ fibroblasts are injected in about 1 ml of suspension.

17. The method of claim 10, in which the suspension is formed by scraping the fibroblasts and/or exposing the fibroblasts to a proteolytic enzyme and/or an animal product free alternative

18. The method of claim 12, wherein between 10⁶ and 10⁸ fibroblasts are injected.

19. The method of claim 18, wherein between about 1 to 5×10⁷ fibroblasts are injected.

20. The method of claim 19, wherein between 1×10⁷ and 4×10⁷ fibroblasts are injected.

21. A composition for repair of a skin defect and/or soft tissue defect in a subject, which comprises a suspension of autologous dermal fibroblasts in a preservation medium having a pH of 7.6 and osmolality of ˜360, and comprising the following components: 6-hydroxy-2,5,7,8-tetramethylchromane-2-carboxylic acid, Na⁺, K⁺, Ca²⁺, Mg²⁺, Cl⁻, H₂PO⁴⁻, HCO³⁻, HEPES, Lactobionate, Sucrose, Mannitol, Glucose, Dextran-40, Adenosine and Glutathione.

22. The composition of claim 21, wherein the fibroblasts have been suspended in the preservation medium at a temperature of about 2° C. to about 8° C. for at least one hour.
